(19) United States
(12) Patent Application Publication
(10) Pub. No.: US 2003/0082106 A1
(43) Pub. Date: May 1, 2003

(54) Magnetic resonance imaging using contrast agents bioactivated by enzymatic cleavage

(76) Inventors: Aleksandr Nivorozhkin, West Roxbury, MA (US); Thomas J. McMurry, Winchester, MA (US); Andrew Kolodziej, Winchester, MA (US)

Correspondence Address: FISH & NEAVE, 1251 AVENUE OF THE AMERICAS, 50TH FLOOR, NEW YORK, NY, 10020-1105, US

(21) Appl. No.: 10/200,477

(22) Filed: Jul. 19, 2002

Related U.S. Application Data
(63) Continuation of application No. PCT/US01/02221, filed on Jan. 22, 2001.
(60) Provisional application No. 60/177,580, filed on Jan. 22, 2000.

(51) Int. Cl. C07K 14/00; A61K 49/00

(52) U.S. Cl. 424/9.34; 530/324

(57) ABSTRACT

The present invention relates to contrast agents for diagnostic magnetic resonance imaging. In particular, this invention relates to novel compounds which exhibit surprisingly improved relaxivity due to improved binding of an amino acid targeting group within the molecules to proteins following specific cleavage of the agent by a peptidase. This invention also relates to pharmaceutical compositions comprising these compounds and to methods of using the compounds and compositions for contrast enhancement during magnetic resonance imaging.

### TECHNICAL FIELD OF THE INVENTION

The present invention relates to contrast agents for diagnostic magnetic resonance imaging. In particular, this invention relates to novel compounds which exhibit surprisingly improved relaxivity due to improved binding of an amino acid targeting group within the molecules to proteins following specific cleavage of the agent by a peptidase. This invention also relates to pharmaceutical compositions comprising these compounds and to methods of using the compounds and compositions for contrast enhancement during magnetic resonance imaging.

### BACKGROUND OF THE INVENTION

Diagnostic and therapeutic imaging techniques, such as magnetic resonance imaging (MRI), may utilize contrast agents to improve the contrast of the images. These agents alter the inherent tissue response to magnetic fields and consequently increase the contrast between tissues in the image. Improving the effectiveness of contrast agents offers the promise of greater sensitivity in the detection of tissues or tissue defects.

Contrast agents utilize a variety of materials to improve the contrast of magnetic resonance images. For example, complexes between gadolinium or other paramagnetic ions and organic ligands are widely used to enhance and improve contrast. The gadolinium complexes increase contrast by increasing the nuclear magnetic relaxation rates of protons found in the water molecules that are accessible to the contrast agents during MRI [Caravan, P., Ellison, J. J., McMurry, T. J., and Lauffer, R. B. (1999) Chem. Rev. 99: 2293]. The relaxation rate of the protons in these water molecules increases relative to protons in other water molecules that are not accessible to the contrast agent. This increase in relaxation rate, or relaxivity, within a specific population of water molecule protons results in an ability to collect more image data in a given amount of time. This in turn results in an improved signal to noise ratio and improved contrast in the image.

It has previously been established that the motion of the contrast agent must be limited in order to achieve maximal increases in relaxivity and correspondingly the optimal improvement in image contrast using this approach. Thus, relaxivity increases may be attained by limiting the tumbling motion of the entire molecule [Lauffer, R. B. (1987) Chem. Rev. 87: 901-927].

One method that has been used to increase the relativity of MRI contrast agents by restricting rotational motion utilizes large molecules or rigid frameworks to which multiple chelated ions are attached [Shukla, R. et al., (1996) Mag. Reson. Med. 35: 928; Shukla, R. B., et al., (1997) Acta Radiol. 412: 121; Ranganathan, R. S., et al., (1998) Invest. Radiol. 33: pp. 779; Jacques, V., et al., (1997) J. Alloys Cmpd. 249: 173]. The attached large molecule slows the molecular motion of the contrast agent and correspondingly increases the relativity of the contrast agent. The images that are generated using these contrast agents, however, suffer from high, undesirable background due to the high signal and relativity of the contrast agents themselves.

Another method for accomplishing this motion limitation is Receptor Induced Magnetization Enhancement (RIME). This method has produced a new generation of gadolinium-chelated imaging agents where the agent has a molecular motion which is decreased when the agent is bound after administration. The RIME technique limits the motion of the contrast agent molecule by restricting its motion when it binds to a target receptor or protein. This method has the advantage that the increase in signal intensity due to increased relaxivity occurs only upon binding to the target protein, so undesirable background signal is minimized.

Contrast agents with even a single chelated ion can be effectively immobilized by noncovalent binding to a target protein using the RIME principles. This noncovalent binding serves to specifically increase the contrast agent's relaxivity upon binding to a target protein. This approach led to the discovery of the first gadolinium-based blood pool contrast agent, MS-325, which was in Phase III clinical trials for noninvasive angiography at the time of this application [Lauffer, R. B.; Parmelee, D. J.; Dunham, S.; Ouellet, H. S.; Dolan, R. P., Witte S.; McMurry, T. J.; Walovich, R. C. (1998) Radiology 207: 529]. In the bloodstream, this contrast agent is noncovalently bound to human serum albumin (HSA). This interaction increases the relaxivity of the protein-bound form of the contrast agent seven to eight times compared to the unbound contrast agent in aqueous solution by slowing molecular rotation. Another advantage is that extravasation from blood vessels into the surrounding tissue is greatly reduced. MS-325 has been described in detail in international patent application WO 96/23526 herein incorporated by reference in its entirety.

There remains a need for an improved mechanism, however, to effectively control the increase in relaxivity upon binding of contrast agents at specific times and locations in vivo, which allows for specific activation of the contrast agent. The greater the number of mechanisms and the more efficient the mechanism for controlling the activation of the contrast agents, the wider the range of potential applications for which the contrast agents may be used. Contrast agents that can be activated at specific locations within the body and that can be activated at specific points in time have the advantage that unwanted background signal is reduced or eliminated.

One inventor has disclosed contrast agents that are not targeted but can become activated at a physiological target (WO 96/38184 invented by Thomas Meade and hereinafter referred to as “Meade”). However, the Meade activation method is based on a completely different principle than that of the present invention. The blocking moiety of the Meade invention prevents water protons from interacting with coordination sites on a metal ion. Agents of the Meade invention that contain the blocking moiety have no coordination sites available (or a partially available site as a result of dynamic equilibrium) for interaction with water protons. Therefore, activation only occurs when the blocking moiety is removed allowing more facile access of water molecules to the inner-sphere coordination sites on the metal complex. Increased exchange at these coordination sites allows the agents to enhance the contrast of tissue near water protons.

The masking polypeptide of the present invention decreases the protein binding affinity of the prodrug compared to its bioactivated contrast agent. A critical feature of the contrast agents of the present invention is the correlation of relaxivity increase and the noncovalent binding of the contrast agent to a target. Additionally, binding to the target is specific and can only occur when the masking polypeptide is removed by a peptidase. This cleavage transforms the prodrug into an active contrast agent that binds the target and exhibits increased relaxivity following binding.

Similarly, WO 97/36619 incorporated herein by reference in its entirety (invented by Randy Lauufer and others and hereinafter referred to as “Lauffer”) discloses bicactivated MRI contrast agents. However, the Lauffer compounds are distinguished because the present compounds contain a masking polypeptide that can be cleaved by a peptidase and a targeting group that is a peptide or amino acid. That is, the present contrast agents have the great advantage that a substantial portion of any molecule can be synthesized by automated solid-phase peptide synthesizers. This provides ease of synthesis and provides a general synthetic scheme that can be adapted for the synthesis of a large number of diverse contrast agents.

### DETAILED DESCRIPTION OF THE INVENTION

The compounds and compositions of the present application are prodrug forms of targeted contrast agents that contain cleavable chemical groups that reduce binding of the contrast agent to a target molecule. The uncleaved prodrug has a low affinity for the target protein and correspondingly exhibits a low relaxivity. Activation of the prodrug occurs by enzymatic cleavage. The activated contrast agent binds to the target molecule to form an active complex of the contrast agent and target. The active contrast agent exhibits relaxivity after binding to the target protein that is several-fold higher than the uncleaved prodrug. This increased relaxivity produces improved contrast in the MRI image. In an example of a preferred embodiment, the target protein is human serum albumin (HSA), the binding group is a substituted phenyl, and the cleavable group is polylysine. Following cleavage of the polylysine, the contrast agent binds tightly to HSA, and the tightly bound complex of activated contrast agent and target exhibits increased relaxivity which results in improved signal and image contrast.

In the present invention, the relaxivity (r₁) of the uncleaved contrast agent is preferably 80% or less than the r₁ of the activated agent. More preferably the r₁ relaxivity is 50% or less of the r₁ relaxivity of the activated agent, more preferably 20% or less, and most preferably 10% or less.

I. Definitions

The term “alkyl,” as used herein alone or as part of another group, denotes optionally substituted, linear and/or branched chain saturated hydrocarbons such as methyl, ethyl, propyl, isopropyl, butyl, isobutyl, tert-butyl, and other groups that are well known in the art.

The term “cycloalkyl,” as used herein alone or as part of another group, denotes optionally substituted, saturated cyclic hydrocarbon ring systems, such as cyclopropyl, cyclobutyl, cyclopentyl, and other groups that are well known in the art.

The compounds and compositions of this invention include pharmaceutically acceptable derivatives thereof. “Pharmaceutically acceptable” means that the compound or composition can be administered to an animal without unacceptable adverse effects. A “pharmaceutically acceptable derivative” means any pharmaceutically acceptable salt, ester, salt of an ester, or other derivative of a compound of this invention which, upon administration to a recipient, produces (directly or indirectly) a compound of this invention or an active inhibitory metabolite or residue thereof. Particularly favored derivatives are those that increase the bioavailability of the compounds of this invention when such compounds are administered to a mammal (e.g., by allowing an orally administered compound to be more readily absorbed into the blood) or that enhance delivery of the parent compound to a biological compartment (e.g., the brain or lymphatic system) relative to the rest of the body.

Pharmaceutically acceptable salts of the compounds of this invention also include cations and anions derived from pharmaceutically acceptable inorganic and organic bases and inorganic and organic acids, as known in the art.

Relaxivities R₁ and R₂, defined as the increase in 1/T₁ or 1/T₂, respectively, per mM of metal ion, measure the ability of a contrast agent to enhance the relaxation rate of spectroscopic or imaging nuclei. Relaxivity units are mM⁻¹s⁻¹.

The compounds of this invention may contain one or more asymmetric carbon atoms and thus may occur as racemates and racemic mixtures, single enantiomers, diastereomeric mixtures and individual diastereomers. All such isomeric forms of these compounds are expressly included in the present invention. Each stereogenic carbon may be of the R or S configuration.

Combinations of substituents and variables envisioned by this invention are only those that result in the formation of stable compounds. The term “stable,” as used herein, refers to compounds that do not deteriorate during manufacture and or a sufficient period of time thereafter. Such compounds are therefore suitable for the purposes detailed herein (e.g., therapeutic, diagnostic, or prophylactic administration to an animal or for use in affinity chromatography applications). Typically, such compounds are stable at a temperature of 40° C. or less, in the absence of moisture or other chemically reactive conditions, for at least a week.

It should also be understood that the compounds of this invention may adopt a variety of conformational and ionic forms in solution, in pharmaceutical compositions and in vivo. Although the depictions herein of specific preferred compounds of this invention are of particular conformations and ionic forms, the disclosure of the invention is not so limited.

II. Structure of the Contrast Agent

The compounds of the present invention comprise at least three domains:

(a) a paramagnetic metal and chelating ligand backbone,

(b) an optional linker,

(c) an amino acid targeting group (preferably directed toward a protein target), and

(d) a covalently attached, cleavable polypeptide which prevents binding to the target. The cleavable group is cleaved following administration of the agent, either by an endogenous protease, or following administration of an exogenous protease.

According to one embodiment, the instant invention provides compounds comprising

(a) a paramagnetic metal chelate backbone structure comprising a chelating ligand and a paramagnetic metal ion, wherein the chelating ligand is selected from the group consisting of diethylenetriamine pentaacetic acid (DTPA), 1,4,7,10-tetraazacyclododecane-tetraacetic acid (DOTA), ethylene diamine tetraacetic acid (EDTA), and 1,4,7,10-tetraazacyclododecane-1,4,7-triacetic acid (DO3A); and wherein the chelating ligand forms a complex with one or more paramagnetic metal ions selected from the group consisting of metal ions with atomic numbers 13, 21-34, 39-42, 44-50, and 57-83;

(b) an optional linker;

(c) a protein binding group comprising an amino acid wherein the amino acid side chain is comprised of from one to three phenyl rings, and wherein each phenyl group is optionally substituted with up to five substitutents selected from the group Z; wherein

Z consists of halogen, CN, NO₂, CF₃, OCF₃, OH, S(C₁-C₄)-alkyl, SO(C₁-C₄)-alkyl, SO₂(C₁-C₄)-alkyl, NH₂, NH(C₁-C₄)-alkyl, N((C₁-C₄)-alkyl)₂, COOH, C(O)O(C₁-C₄)-alkyl, O(C₁-C₄)-alkyl; (C₁-C₆)-alkyl, (C₂-C₆)-alkenyl, (C₂-C₆)-alkynyl, and (C₃-C₇)-cycloalkyl, and

(d) a covalently attached, cleavable masking polypeptide wherein the masking polypeptide comprises from one to ten amino acid residues residues, and wherein the cleavage site is a peptide bond.

A particularly preferred chelating ligand comprises a pharmaceutically acceptable metal chelate compound consisting of one or more cyclic or acyclic organic chelating agents complexed to one or more metal ions. Paramagnetic metal ions preferred for MRI include those with atomic numbers 21-29, 42, 44, or 57-83.

The paramagnetic metal ion should not dissociate from the chelating ligand to any significant degree during the imaging agent's passage through the body, including passage through a tissue where the contrast agent may undergo biomodification. Significant release of free metal ions can result in large MRI alterations and may also be accompanied by toxicity, which would only be acceptable in pathological tissues. Preferably bioactivation does not significantly compromise the stability of the metal-chelate complex and the metal remains intact and is excreted.

In general, the degree of toxicity of a metal chelate is related to its degree of dissociation in vivo before excretion. Toxicity generally increases with the amount of free metal ion; that is, a high formation constant is preferred to prevent toxic concentrations of free metal ions. Particularly preferred are formation constants of at least 10¹⁵ M⁻¹, or at least 10¹⁶ M⁻¹, or at least 10¹⁷ M⁻¹, or at least 10¹⁸ M⁻¹, or at least 10¹⁹ M⁻¹, or at least 10²⁰ M⁻¹, or at least 10²² M⁻¹ or at least 10²⁴ M⁻¹ or higher. If the kinetics of metal ion dissociation are very slow, then a complex having a lower formation constant, i.e. of at least 10¹⁰ M⁻¹, may be sufficient.

The preferred paramagnetic metal is selected from the group consisting of Gd³⁺, Fe³⁺, Mn²⁺, Mn³⁺, Cr³⁺, Cu²⁺, Dy³⁺, Tb³⁺, Ho³⁺, Er³⁺ and Eu³⁺. The most preferred metal is Gd³⁺. Many suitable chelating ligands for MRI agents are known in the art. These can also be used for metal chelates for other forms of biological imaging. For MRI imaging, preferred chelating ligands include but are not limited to derivatives of:

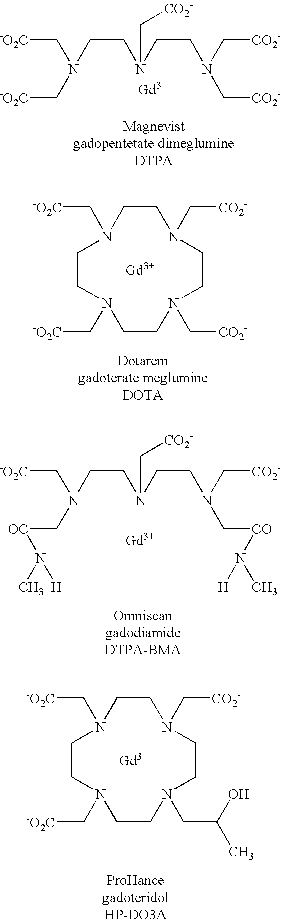

Although these chelating ligands are all shown with a chelated Gd³⁺, it is known in the art that other metals may be substituted for Gd³⁺ in certain applications.

The present application describes novel compounds comprising gadolinium complexes with poor HSA binding and low relaxivity which can be transformed via enzymatic cleavage to species with improved HSA binding and enhanced relaxivity. The compounds of this invention may be modified so that cleavage is accomplished by specific proteases that have been identified as useful targets in disease diagnostics and treatment.

One attribute of the present invention is a binding group that comprises an amino acid side chain. Such a binding group allows simplified synthesis of the contrast agent since the agent can be synthesized using standard peptide synthesis techniques.

Another attribute of the present invention is a cleavable group comprised of amino acids. Again this allows simplified synthesis of the contrast agent since standard peptide synthesis techniques are used. The amino acids may be chosen for their ability to prevent binding of the contrast agent to the target. In a particularly preferred embodiment, the cleavable polypeptide comprises positively charged amino acids. The amino acids of the cleavable group may also be chosen based on the specificity of the protease that cleaves the amino acids. Other factors may influence the selection of the amino acids that comprise the binding group and cleavable polypeptide.

Another particular attribute of the present invention is the structure of the amino acid targeting group. Many targeting groups may bind a particular target molecule. The targeting group is an amino acid that is covalently bound to the chelating ligand either directly or via a short linker. A preferred embodiment of the present invention comprises binding groups that bind to protein targets. Particularly preferred are amino acids that comprise one or more aromatic groups, preferably phenyl groups. More preferred are amino acids that comprise phenyl groups substituted with one to five groups selected from the group Z consisting of halogen, CN, NO₂, CF₃, OCF₃, OH, S(C₁-C₄)-alkyl, SO(C₁-C₄)-alkyl, SO₂(C₁-C₄)-alkyl, NH₂, NH(C₁-C₄)-alkyl, N((C₁-C₄)-alkyl)₂, COOH, C(O)O(C₁-C₄)-alkyl, O(C₁-C₄)-alkyl; (C₁-C₆)-alkyl, (C₂-C₆)-alkenyl, (C₂-C₆)-alkynyl, and (C₃-C₇)-cycloalkyl.

An optional linker may connect the chelating ligand and the targeting amino acid. Preferred linkers are relatively short and limit the mobility of the chelate, especially when bound to HSA. Most preferred are linkers that are no more than six atoms in length and link atoms that comprise the amino acid targeting group and the chelate. Preferred linkers are a carbonyl, glycine, or both taken together.

The preferred embodiments of the present invention contain a cleavable polypeptide group that is cleaved in vivo. Preferred embodiments comprise cleavable groups that are cleaved by an enzyme selected from the Thrombin Activatable Fibrinolysis Inhibitor (TAFI), a member of the Carboxypeptidase B family, trypsin, Factor Xa, 7B2 protein, proprotein convertase 2, subtilisin, kexin endoproteinase, pancreatic carboxypeptidase, Endoproteinase Lys-C, Myxobacter Protease, elastase, matrix metalloproteinases (MMPs), Clostripain, and Armillaria Protease. The invention further contemplates the use of other enzymes known to site-specifically cleave peptides, such as chymotrypsin, especially when the masking polypeptide includes positively charged terminal amino acids. The most preferred embodiments comprise cleavable groups that are cleaved by the proteolytic enzyme TAFI, a member of the Carboxypeptidase B class of proteolytic enzymes. TAFI acts in vivo by cleaving C-terminal lysines exposed on fibrin. After fibrin is cleaved in vivo, clot degradation by tissue plasminogen activator and plasminogen is inhibited. Following cleavage of the contrast agents of the present invention by the TAFI enzyme, the contrast agents bind more tightly to the target protein resulting in increased relaxivity and improved image contrast.

Screening of a large number of candidate contrast agents has previously shown that incorporating aryl groups into the structure of traditional gadolinium polyaminocarboxylate ligands, such as DOTA or DTPA, results in improved binding of the contrast agents to HSA. To maximize relaxivity, binding groups should not be placed more than about 20 carbon-carbon bond lengths from the metal center since the additional intervening atoms provide additional flexibility to the molecular structure, which in turn may allow increased, undesirable molecular tumbling or increased motion of the chelated paramagnetic metal ion at the chelation site. Any decrease in molecular tumbling or chelate motion will result in increases in relaxivity. Therefore, the linker between the chelation ligand and the targeting amino acid should be relatively short.

Contrast agents containing a masking polypeptide comprising positively charged amino acids (e.g., lysine, arginine, ornithine, 2,4-diaminobutanoic acid, 2,3-diaminopropionic acid or other residues) bind less tightly to HSA and exhibit lower relaxivity in aqueous media containing HSA than contrast agents lacking positively charged amino acids. Positive charges significantly attenuate the affinity of the molecule for HSA. Cleavage of the charged amino acids by an appropriate enzyme (e.g., TAFI which cleaves polylysine), therefore permits the contrast agent to bind more tightly to HSA. Tight binding of the contrast agent to HSA results in increased relaxivity. The peptide is preferably covalently attached to the linker/chelate via its N-terminus. This leaves the negatively charged C-terminus exposed and allows the peptide to be cleaved by a carboxypeptidase. After such cleavage and removal of positively charged amino acids, the remaining negatively charged carboxylate group may facilitate binding of the “unmasked” agent to HSA.

III. Examples

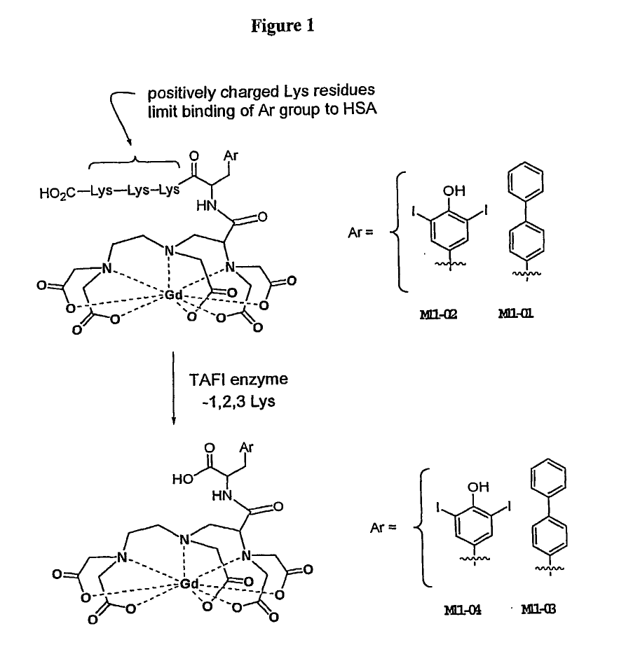

In a particular embodiment, modified DTPA-chelating ligands containing Gd³⁺ complexed at the chelation site are conjugated to an amino acid wherein the side chain is either diphenylalanine or 3,5-diiodotyrosine, both of which bind well to HSA. The Gd³⁺ chelate serves as a signaling domain and is coupled to an HSA binding moiety masked by an HSA masking polypeptide (preferably polylysine) that inhibits binding to HSA. Enzymatic cleavage releases the masking polypeptide group and activates the contrast agent by promoting HSA binding. FIG. 1 shows the structure of the diphenylalanine (M11-01) and 3,5-diiodotyrosine (M11-02) compounds. The addition of multiple charged groups, such as lysine residues, to the amino acid targeting groups inhibits binding to HSA since charged groups are known to bind poorly to HSA. The lysine residues of M11-01 and M11-02 can be cleaved by TAFI to yield compounds M11-03 and M11-04, respectively. Cleavage of the lysine residues results in activation.

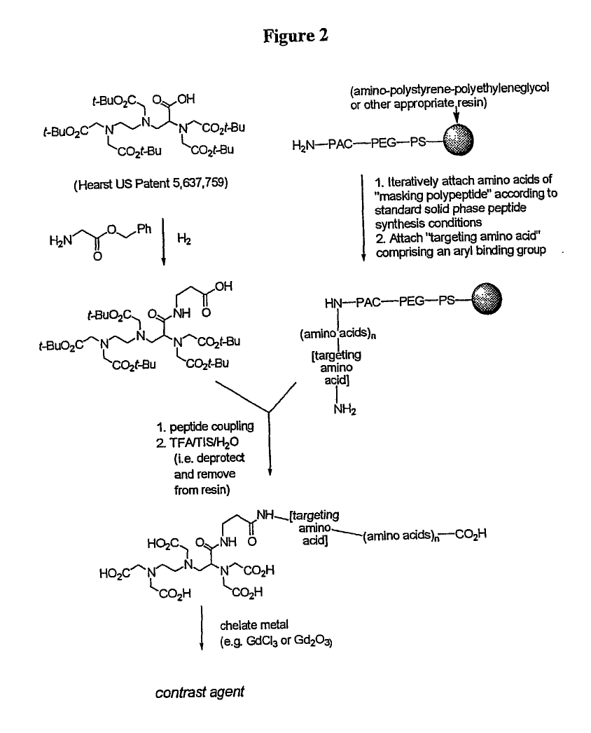

The synthesis of the preferred embodiments, M11-01 and M11-02, is shown in FIG. 2. The left side of the scheme shows the synthesis of the glycine conjugated diethylenetriamine pentaacetic acid (DTPA) derivative. The starting material is a t-butyl ester of the carboxylated DTPA, the synthesis of which is described in U.S. Pat. No. 5,637,759 (the “Hearst” patent). The “Hearst” patent also describes the synthesis of a related EDTA analog. Conversion of the carboxylated t-butyl ester of DTPA to the glycine conjugate is accomplished by reaction with the benzyl ester of glycine under standard peptide coupling conditions followed by hydrogenation to remove the benzyl protecting group.

The right side of the scheme shows the parallel synthesis of the masking polypeptide and targeting amino acid on a solid phase support such as that employed by automated peptide synthesizers. The amino acids attached to the solid phase support are coupled to the carboxylated glycine conjugate of the DTPA t-butyl ester. The carboxylate groups are deprotected and the contrast agent precursor is removed from the solid phase support as shown. The active contrast agent is then synthesized by addition of the metal which is chelated by the DTPA derivative.

Peptide synthesis may be in either the N-to-C or C-to-N direction, and the linker/chelate may be attached to either end of the peptide. Preferably, peptides are attached at the N-terminus during synthesis and the resin preferably produces a free carboxylate at the C-terminus.

The linker, when present, may be coupled to the growing polypeptide chain that is attached to the resin using conventional solid phase peptide synthesis. Amine-containing linkers are preferably attached to the C-terminus of the peptide, and carbonyl-containing linkers are preferably attached to the N-terminus of the peptide since a peptide bond will result in either case. The DTPA and EDTA chelates of U.S. Pat. No. 5,637,759 (the “Hearst” patent) are particularly preferred since those compounds may be attached to the peptide using ordinary solid phase peptide synthesis. For syntheses using these compounds, higher yields are achieved when a glycine linker is inserted between the chelate and the targeting amino acid. In this embodiment, the linker is —C(O)-gly-, wherein the carbonyl is derived from the compounds in the aforementioned Hearst patent, and the glycine residue is added to the peptide attached to the solid phase.

Synthetic routes to other compounds are also illustrated in FIG. 2. For example, the linker conjugated to the peptide may be attached to an acetate group of the chelating ligand. The bond to the acetate group may be formed either directly to the carbonyl of the acetate (for example, the acetate carbonyl may form a peptide bond with an amine group linker-conjugated peptide) or indirectly via the carbon atom a to said carbonyl.

Preferred linkers are relatively short and limit the mobility of the chelate, especially when bound to HSA. Most preferred are linkers that are no more than six atoms in length and link atoms that comprise the amino acid targeting group and the chelate.

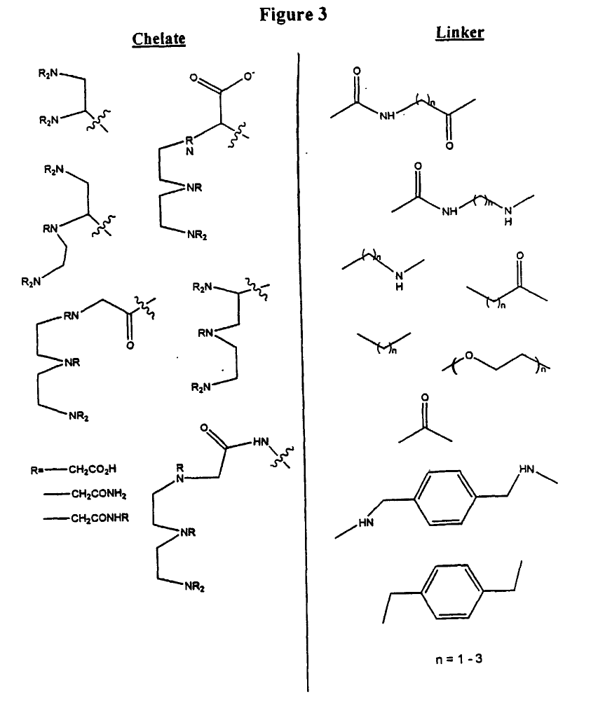

The linker, when present, connects the metal chelating complex with the targeting amino acid, and its principal purpose is to facilitate the synthesis of the compounds. Example linkers include linear, branched, or cyclic alkyl groups, aryl groups and heterosubstituted analogs, for example, ethers, amines, amides. In general, the smallest linker which allows the proper expression of target binding and highest relaxivity is preferred. In the case of linear alkyl linkers, less than 10 atoms are preferred, less than 6 atoms more preferred, and less than 4 atoms most preferred. In some cases, heterosubstituted linkers are preferred for convenient synthesis or desirable physical properties. For example, a short amino acid sequence (e.g., Ala, Gly, Gly-Gly or Ala-Ala) may provide optimal spacing between the metal complex and the targeting amino acid. Some examples of linkers are shown in FIG. 3.

In addition to the linkers described in FIG. 3, other preferred linkers comprise 1 to 6 atoms and are linear and unbranched. The linkers preferably connect the chelating group (for example, via a carbon of the ethylene backbone or a carbon of an acetate group) and the alpha-nitrogen or carbonyl carbon of the amino acid targeting group. The linker portion of the molecule is preferably derived, either in whole or in part, from the synthetic chemistry intermediates that terminate in an amine or a carboxylate (including both protected and activated forms thereof) which are compatible with the synthetic chemistry conditions typically used for solid phase peptide synthesis. Finally, carbon atoms of the linkers may optionally be substituted with hydroxyl groups or halogens.

The solid phase resin used for the synthesis of the DTPA-peptide conjugates is polyethylene glycol-polystyrene (PEG-PS) resin with a [3-(4-hydroxymethylphenoxy)acetic acid] (PAC) handle and fluorenylmethyloxy carbonyl (fmoc) protected amino acids. This method utilizes the coupling agent O-(7-azabenzotriazol-1-yl)-1,1,3, 3-tetramethyluronium hexafluorophosphate (HATU) in the presence of diisopropylethyl amine (DIPEA) for stepwise attachment of the individual amino acids comprising the DTPA-peptide molecule to the support structure according to the standard practice known in the art. The resultant molecule is cleaved from the resin and the t-butyl esters are converted to the free carboxylic acids moieties with a mixture of trifluoroacetic acid (TFA)/triisopropylsilane (TIS)/H₂O for 2 h.

The products are purified by reversed phase high performance liquid chromatography (HPLC) on a C₁₈ column using a linear gradient of 0.1% TFA in acetonitrile and 0.1% aqueous TFA. Gd⁺³ complexes are obtained in aqueous solution at pH 3.0-7.5, preferably between pH 4.5 and 5.5 in acetate buffer, by mixing the contrast agent precursor with GdCl₃. The final Gd⁺³ concentrations are assessed by Inductively Coupled Plasma (ICP) atomic emission spectroscopy. The identity and purity of both the ligands and complexes were confirmed by electrospray-mass spectroscopy (ES-MS) and liquid chromatography-mass spectroscopy (LC-MS).

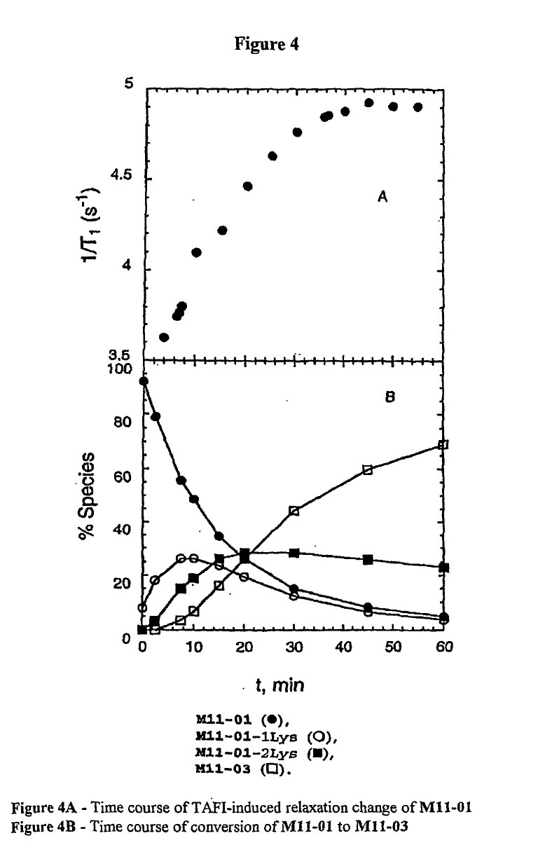

FIG. 4 shows the time course for cleavage of the polylysine residues from the preferred compound M11-01. FIG. 4A shows the time course of the TAFI-induced increase in 1/T₁ that accompanies the cleavage of the polylysine residues from the M11-01 compound to form M11-03. The increase in 1/T₁ was measured at a magnetic field strength of 0.5 T and a reaction temperature of 24° C. The initial concentration of M11-01 was 0.2 mM, and the reaction was carried out in the presence of 4.5% (w/v) HSA and 75 nM of the TAFI enzyme.

FIG. 4B shows the time course for the conversion of M11-01 to M11-03 by cleavage of the lysine residues. The percentage of the species present is plotted on the ordinate axis versus the time in minutes. Cleavage is not 100% efficient; thus, some amount of the partially cleaved M11-01 compound remains after 60 minutes. M11-01 designates the compound with three lysine residues, M11-01-2Lys designates the partially cleaved product that still contains two lysines residues (one lysine has been cleaved) while M11-01-2Lys is the partially cleaved product containing one lysine residue (two lysines have been cleaved). M11-03 is the fully cleaved and fully active contrast agent.

The increases in 1/T₁ most closely coincides with the disappearance of M11-01 (FIG. 4B). Between 0 and 30 min, 1/T₁ increased from 3.6 to 4.8 s⁻¹ while approximately 85% of M11-01 was converted to M11-03 or intermediates. Between 30 and 60 min, 1/T₁ changed only slightly (from 4.8 to 5.0 s⁻¹) while the concentration of M11-03 increased 55% from 100 to 160 μM. Removal of the two C-terminal lysine residues resulted in the bulk of the 1/T₁ increase, whereas removal of the third lysine appeared to be less critical for achieving a significant effect.

The progress of enzymatic turnover by TAFI at 24° C. was followed by HPLC and quantified by the peak integration of the ligand form of the substrate and reaction products. HPLC analysis of the reaction mixture, quenched at various time points, confirmed the production of the dilysine and monolysine intermediates and M11-03. TAFI was activated prior to substrate addition for 10 min at room temperature in the following conditions: 250 nM TAFI, 10 nM a-thrombin, 25 nM thrombomodulin in 10 mM HEPES, 150 mM NaCl and 5 nM CaCl₂. TFA was used for the reaction quenching. No influence of HSA on the enzyme kinetics was revealed. HSA binding percentage was determined by ultrafiltration.

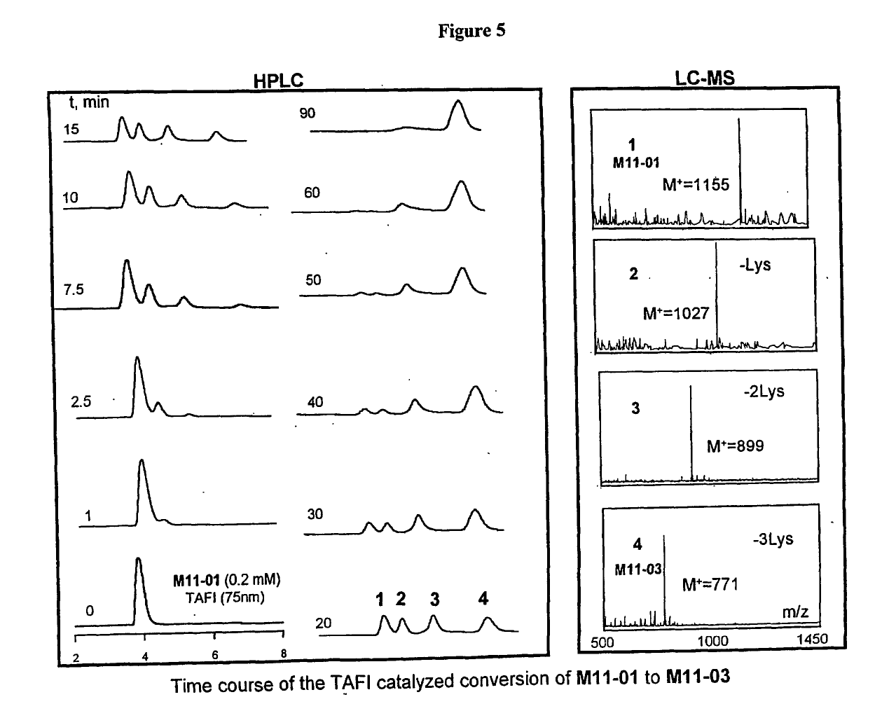

Enzyme kinetics were studied in more detail for one of the compounds, M11-01. FIG. 5 shows the HPLC and the mass spec (LC-MS) profiles for the four compounds (M11-01, M11-01-1Lys, M11-01-2Lys, and M11-03 each of which successively contains one less lysine). The left side of the figure shows the relative concentrations of the four compounds as determined by HPLC during enzymatic cleavage between 0 and 60 minutes. This part of the figure shows that M11-01 is almost exclusively present at the beginning of the experiment and M11-03 (“labeled-3Lys”) is almost exclusively present at the end of the experiment. The right part of the figure shows the LC-MS profile for the four compounds. The mass to charge ratio (m/Z) is plotted on the abscissa. The figure shows that the mass of the compounds decreases proportionately with the loss of the lysine residues.

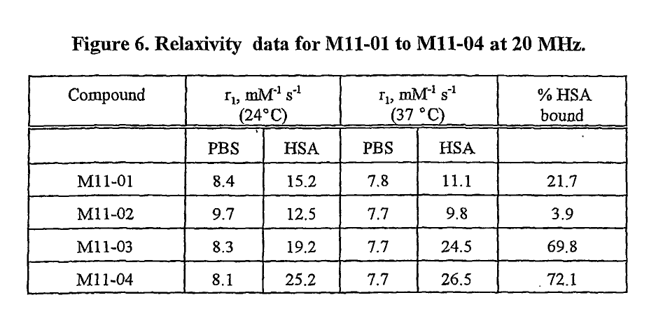

The data on the relaxivity and HSA binding of the preferred uncleaved compounds (M11-01 and M11-02) as well as the final products of the enzymatic reaction (M11-03 and M11-04) are presented in FIG. 6 and illustrated in FIG. 1. The Table shows the relaxivities at 24° C., at 37° C., and the percentage of the compounds that are bound to HSA at 37° C.

At 24° C., an increase in relaxivity is observed for the cleavage of both M11-01 and M11-02 to form M11-03 and M11-04, respectively. The cleavage of M11-01 results in a 26% increase in relaxivity while the cleavage of M11-02 results in more than a 100% increase in relaxivity. At 37° C., a 120% increase in relaxivity results from cleavage of M11-01 while a 170% increase in relaxivity results from cleavage of M11-02. No increase in relaxivity is observed for cleavage of the compounds in phosphate buffered saline (PBS), which lacks HSA, at either temperature.

A second compound, M11-02 exhibited an improved relaxivity increase following cleavage of the polylysine residues compared to M11-01. Relaxivities of M11-02 and the non-lysine compound M11-04 in the presence of 4.5% HSA at 24° C. were 12.5 and 25.2 mM⁻¹ s⁻¹, respectively. Complete conversion of M11-02 to M11-04 by TAFI was achieved at micromolar enzyme concentration and generated an expected 100% relaxivity enhancement due to the observed 18-fold increase in HSA binding activity. A time course of the TAFI reaction at nanomolar level (200 μM 2, 75 nM TAFI, 4.5% w/v HSA) monitored by the 1/T₁ change yielded a smaller effect due to the slower cleavage of the third lysine residue which competed with TAFI autoinactivation. At 30 min, the monolysine intermediate represented 83% of all the species whereas M11-04 accounted for 5% of the total. In contrast to M11-01, removal of the third lysine turned out to be essential for attaining the maximal 1/T₁ increase, however, the full realization of maximum relaxivity may not be necessary for clinical utility. At the endpoint of the turnover by TAFI, the monolysine intermediate and M11-04 represented 44% and 51% of the reaction mixture, respectively, but 1/T₁ had only increased by 26% to 3.83 s⁻¹. This indicates that M11-02 must be exposed to TAFI for a longer period of time in order to achieve beneficial signal enhancement. However, relaxivity enhancement as high as almost 3-fold can ultimately be reached at 37° C. (FIG. 6).

The relaxivity (r₁) was calculated from the slope of the plot of 1/T₁ vs. sample concentration according to the equation:

$${r}_{1} = \frac{\frac{1}{{T}_{1}} ( Gd solution ) - \frac{1}{{T}_{1}} ( pure buffer )}{conc . of Gd solution}$$

Relaxivity units are mM⁻¹s⁻¹ as shown. The T₁ values were measured at 20 MHz by standard inversion-recovery methods.

The percentage of each compound that is bound to HSA is shown in the right column of FIG. 6. The percentage bound to HSA increases following cleavage for both of the compounds. The cleavage of M11-01 results in a 220% increase in the percentage bound to HSA while the cleavage of M11-02 results in more than a 180-fold increase in the percentage bound to HSA.

At physiological concentrations of TAFI (75 nM), MM11-01 is rapidly converted to M11-03. The kinetic parameters for the disappearance of MM11-01 (Kₘ=340 μM; k_{cat}=5.3 s⁻¹) are comparable to other TAFI substrates such as hippuryl arginine (Kₘ=140 μM; k_{cat}=21 s⁻¹) The time course for turnover of M11-01 (230 μM) by TAFI in the presence of 4.5% HSA was complete within an hour.

We claim:

1. A magnetic resonance imaging contrast agent having the structure: 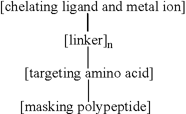
wherein:
the chelating ligand forms a complex with one or more paramagnetic metal ions selected from the group consisting of metal ions with atomic numbers 13, 21-34, 39-42, 44-50, and 57-83;
the linker, if present, comprises from 1 to 20 carbon atoms wherein the carbon atoms form a linear, branched, or cyclic alkyl group, aryl groups, or heterosubstituted analogs thereof wherein from 1 to 6 carbon atoms are replaced by nitrogen, oxygen or sulfur atoms;
n is 0 or 1;
the targeting amino acid comprises from one to three phenyl rings attached as a side chain at the α carbon of the amino acid wherein:
each phenyl group is optionally substituted with up to five substitutents selected from the group Z; wherein
Z consists of halogen, CN, NO₂, CF₃, OCF₃, OH, S(C₁-C₄)-alkyl, SO(C₁-C₄)-alkyl, SO₂(C₁-C₄)-alkyl, NH₂, NH(C₁-C₄)-alkyl, N((C₁-C₄)-alkyl)₂, COOH, C(O)O(C₁-C₄)-alkyl, O(C₁-C₄)-alkyl; (C₁-C₆)-alkyl, (C₂-C₆)-alkenyl, (C₂-C₆)-alkynyl, and (C₃-C₇)-cycloalkyl;
the masking polypeptide comprises from one to ten amino acid residues residues; and wherein
a cleavage site exists between the targeting amino acid and the masking polypeptide and said cleavage site is a peptide bond.

2. The compound according to claim 1 wherein the chelating ligand is selected from tetraamine 1,4,7,10-tetraazacyclododecane-N,N′,N″,N″-tetraacetic acid (DOTA), 1,4,7,10- tetraazacyclododecane-1,4,7-triacetic acid (DO3A), or one of the following structures: 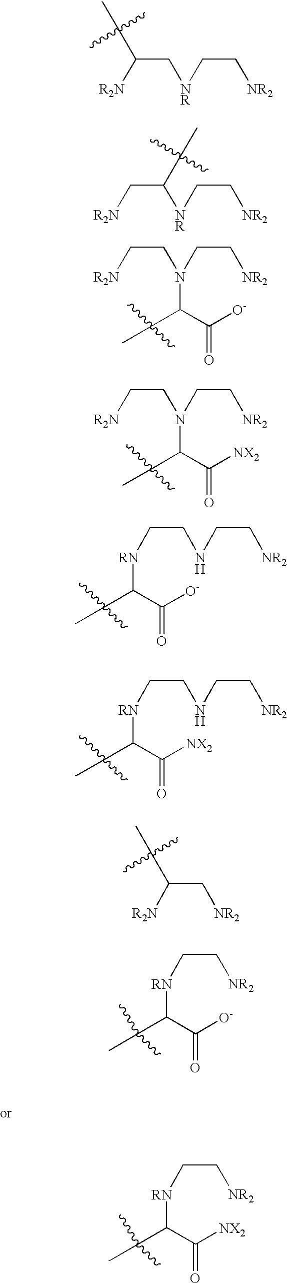
wherein:
R is selected from —CX₂C(O)O— or —CX₂C(O)NX₂;
X is H, (C₁-C₆) straight or branched chain alkyl, (C₂-C₆) straight or branched chain alkenyl, or (C₂-C₆) straight or branched chain alkynyl;
the single bond with the intersecting wavy line is the point of attachment for the linker; and
C(O) indicates a carbon-oxygen double bond.

3. The compound according to claim 2 wherein Z is halogen, CN, NO₂, CF₃, OCF₃, or OH.

4. The compound according to claim 3 wherein the targeting amino acid is 3,5 diiodotyrosine.

5. The compound according to claim 3 wherein said targeting amino acid is a diphenylalanine group.

6. The compound according to claim 3 wherein the paramagnetic metal ion is selected from the group of metals having atomic numbers 21-29, 42, 44 or 57-83.

7. The compound according to claim 6, wherein the paramagnetic metal ion is Gd³⁺.

8. The compound according to claim 7 wherein the chelating ligand and paramagnetic metal ion is gadolinium diethylenetriamine pentaacetic acid (Gd-DTPA).

9. The compound according to claim 3, wherein the metal chelate has a formation constant of greater than about 10²⁰ M⁻¹.

10. The compound according to any of claims 1 to 9, wherein the covalently attached, cleavable masking polypeptide group is removed by an enzyme selected from the group consisting of Thrombin Activatable Fibrinolysis Inhibitor (TAFI), a member of the Carboxypeptidase B family, trypsin, Factor Xa, 7B2 protein, proprotein convertase 2, subtilisin, kexin endoproteinase, pancreatic carboxypeptidase, Endoproteinase Lys-C, Myxobacter Protease, elastase, matrix metalloproteinases (MMPs), Clostripain, and Armillaria Protease.

11. The compound according to claim 10, wherein the covalently attached, cleavable masking polypeptide group is removed by TAFI or another member of the Carboxypeptidase B family.

12. A pharmaceutical composition comprising a contrast agent according to any one of claims 1 to 9, and a carrier, adjuvant or vehicle.

13. A method of MRI imaging comprising the step of administering a diagnostically effective amount of a compound according to any one of claims 1 to 9.

14. A method for MRI imaging comprising the step of administering a diagnostically effective amount of a composition according to claim 12.

15. A method of bioactivating a contrast agent comprising the steps of administering a compound according to any one of claims 1 to 9, and activating the contrast agent via cleavage of the masking polypeptide by a protease.

16. A method for bioactivating a contrast agent comprising the steps of administering a pharmaceutical composition according to claim 12, and activating the contrast agent via cleavage of the masking polypeptide by a protease.
